(19) United States
(12) Patent Application Publication
(10) Pub. No.: US 2005/0233427 A1
(43) Pub. Date: Oct. 20, 2005

(54) Processes for the production of triglycerides of unsaturated fatty acids in the presence of enzymes

(76) Inventors: Ulrich Schoerken, Duesseldorf (DE); Carolin Meyer, Duesseldorf (DE); Sabine Both, Duesseldorf (DE); Peter Horlacher, Bellenberg (DE)

Correspondence Address: COGNIS CORPORATION;PATENT DEPARTMENT, 300 BROOKSIDE AVENUE, AMBLER, PA, 19002, US

(21) Appl. No.: 11/093,534

(22) Filed: Mar. 30, 2005

(30) Foreign Application Priority Data
Mar. 31, 2004 (DE) 10 2004 015 782.0

(51) Int. Cl. C12P 7/64

(52) U.S. Cl. 435/134

(57) ABSTRACT

Processes for the enzyme-catalyzed synthesis of triglycerides containing polyunsaturated fatty acids in which the synthesis is preceded by a pre-hydrolysis in which (a) C₁₋₄ alkyl esters of polyunsaturated fatty acids are completely or partly hydrolyzed in vacuo with continuous addition of water or are partly hydrolyzed in several stages without vacuum, (b) after the hydrolysis, the water is removed by separation or distillation and (c) the synthesis of the polyunsaturated fatty acids with glycerol in vacuo in the presence of an enzyme to form their triglycerides is carried out under low-water conditions, (d) the enzymes are removed from the triglyceride by separation or filtration and (e) the remaining fatty acids and/or possible residues of C₁₋₄ alkyl esters are removed from the triglyceride by distillation or refining; the pre-hydrolysis and the subsequent synthesis preferably being carried out as a one-pot process in the same reactor and in the presence of the same enzyme.

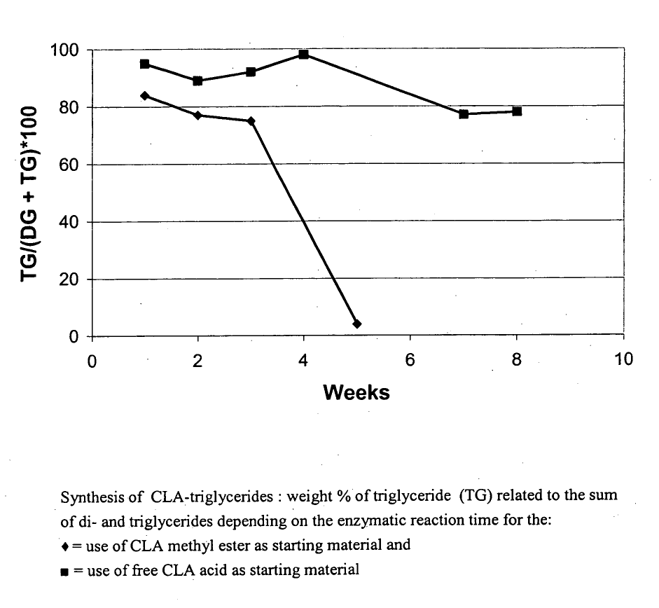

### BACKGROUND OF THE INVENTION

Esters of polyunsaturated fatty acids can be produced both by chemical and by enzymatic methods. Chemical syntheses have the disadvantage that very high temperatures generally have to be used and large quantities of basic catalysts are required, so that secondary products and unwanted isomerizations occur to a fairly significant extent. Enzyme-catalyzed reactions with lipases generally take place under milder conditions and give high-purity end products.

European patent application EP 1 322 776 A1 describes a lipase-catalyzed method for the production of triglycerides of polyunsaturated conjugated fatty acids from the alkyl ester of the unsaturated fatty acids and glycerol which removes the alcohol formed from the reaction under reduced pressure. In addition, International patent application WO 9116443 A1 describes the esterification of glycerol and free polyunsaturated fatty acids or alkyl esters thereof to form the corresponding triglycerides by removing the water of reaction or the alcohol formed under reduced pressure.

However, enzymatic syntheses often have the disadvantage that the reactions are relatively slow. The stability of most enzymes under very low-water conditions, as for example in the direct transesterification of alkyl esters to glycerides, is poor. If the free acid is used, which accelerates the actual triglyceride synthesis, esters of unsaturated fatty acids still have to be hydrolyzed beforehand.

Accordingly, the problem addressed by the present invention was to improve the profitability of enzymatic processes for the production of triglycerides containing polyunsaturated fatty acids.

### SUMMARY OF THE INVENTION

This invention relates generally to fatty acid esters and, more particularly, to a new process for the enzymatic synthesis of triglycerides containing polyunsaturated fatty acids which consists of a pre-hydrolysis of alkyl esters of polyunsaturated fatty acids in an aqueous environment and a reaction of the fatty acids released to form triglycerides under low-water conditions.

The present invention includes a process for the enzyme-catalyzed synthesis of triglycerides containing polyunsaturated fatty acids in which the synthesis is preceded by a pre-hydrolysis in which:
(a) C₁₋₄ alkyl esters of polyunsaturated fatty acids are completely or partly hydrolyzed in vacuo with continuous addition of water or are partly hydrolyzed in several stages without vacuum,
(b) after the hydrolysis, the water is removed by separation or distillation and
(c) the synthesis of the polyunsaturated fatty acids with glycerol in vacuo in the presence of an enzyme to form their triglycerides is carried out under low-water conditions,
(d) the enzymes are removed from the triglyceride by separation or filtration and
(e) the remaining fatty acids and/or possible residues of C₁₋₄ alkyl esters are removed from the triglyceride by distillation or refining.

The pre-hydrolysis (steps a and b) and the subsequent synthesis (steps c to e) are preferably carried out as a one-pot process in the same reactor and, more particularly, in the presence of the same enzyme. The advantage of this process is better stabilization of the enzyme for the triglyceride synthesis.

It has been found that the stability and hence the re-usability of the enzyme are significantly improved and hence the costs of the process can be reduced because the enzyme is generally the most cost-intensive factor of such a synthesis process.

### BRIEF DESCRIPTION OF THE DRAWING

The drawing is a graphical comparison of the synthesis of CLA triglycerides as a function of reaction time using a CLA methyl ester and CLA (free acid) as alternative starting materials.

### DETAILED DESCRIPTION OF THE INVENTION

The drawing illustrates the reaction of CLA methyl ester (♦) or free acid CLA (▪) with glycerol to CLA triglycerides in the presence of the immobilized lipase Novozym 435 (Lipase from Novozymes, Denmark). It was found that, where the free acid was used, there was a smaller reduction in enzyme activity. Thus, after 5 weeks, the Novozym 435 in the batch containing CLA methyl ester could no longer be filtered. However, the use of free acid does presuppose ester cleavage of the CLA predominantly present in the form of esters. If, as known in the prior art, the esters were directly reacted with the enzyme in a transesterification, the enzyme would suffer serious losses of stability (see drawing).

Accordingly, the process according to the invention consists of two steps in which a pre-hydrolysis precedes the actual synthesis. Before the esterification, the CLA alkyl ester is split into alcohol and the free acid in a water-rich medium, preferably in the same reaction vessel. By “water-rich” is meant a quantity of water of at most 50%, preferably at most 25%, and at least 1% and preferably at least 5% water, based on the reaction mixture as a whole. Even in a vacuum, a water content of at least 1% and preferably at least 5%, based on the reaction mixture as a whole, is adjusted in the reaction mixture by the continuous addition of water. The hydrolysis may be carried out by known chemical methods. Preferably, however, the pre-hydrolysis is also carried out in the presence of enzymes, more particularly in the presence of the same enzyme which is used in the subsequent synthesis.

The synthesis of the triglycerides is carried out under low-water conditions. By this meant that the synthesis is carried out in vacuo without the addition of water or steam. Small quantities of water are continuously formed in the synthesis through the formation of the esters from glycerol and fatty acid. This water of reaction protects the lipase against complete dehydration. By contrast, where the esters are used as substrates, no water of reaction is formed, so that dehydration of the lipase and hence deactivation occur more quickly in vacuo. Compared with the chemical synthesis of triglycerides of polyunsaturated fatty acids, the enzymatic synthesis can be carried out at far lower temperatures which leads to a reduction in unwanted secondary products, such as unwanted isomers for example. The reaction rate of this enzymatic process is normally very low. However, the process according to the invention leads to stabilization of the enzymes used which can thus be re-used, so that the enzymatic process is made profitable.

The process is applicable to C₁₋₄ alkyl esters, preferably methyl and/or ethyl esters, selected from the group consisting of naturally occurring polyunsaturated and polyconjugatedly unsaturated fatty acids and conjugated linoleic and linolenic acids. Esters of docosahexaenoic acid, eicosapentaenoic acid, arachidonic acid, γ-linolenic acid and conjugated linoleic acid are preferably used, the c9, t11 and t10, c12 isomers of conjugated linoleic acid (CLA) thereof being particularly preferred. The concentration range selected for the raw materials used is from 3 to 6 mol ester to 1 mol glycerol, 3.2 to 4.0 mol ester to 1 mol glycerol preferably being used to achieve an optimal reaction rate.

Typical examples of suitable enzymes, which are not intended to limit the invention in any way, are lipases and/or esterases of microorganisms selected from the group consisting of Alcaligenes, Aspergillus, Candida, Chromobacterium, Rhizomucor, Penicilium, Pseudomonas, Rhizopus, Thermomyces, Geotrichum, Mucor, Burkholderia and mixtures thereof. Lipases and esterases from the organisms Alcaligenes, Candida, Chromobacterium, Penicilium, Pseudomonas, Rhizopus, Rhizomucor and Thermomyces are preferred because they are particularly active, Candida and Rhizopus and especially Candida antarctica B, being particularly preferred. Lipases immobilized on carrier material are particularly suitable, more especially 3 to 12% by weight of immobilizate, based on the percentage fat content.

The temperature range suitable for the reaction is determined by the optimum activity of the enzymes. Temperatures in the range from 40 to 90° C. have proved to be particularly suitable for the lipases preferably selected, temperatures in the range from 55 to 80° C. being preferred. A vacuum of at least 200 mbar, preferably 1 to 100 mbar and more preferably 20 to 60 mbar should be applied. The preferred process parameters are determined by the acceleration to be achieved in the reaction rate.

On completion of the reaction, the immobilized enzymes are removed by separation or filtration and the unreacted fatty acids or alkyl esters thereof are removed by refining or distillation, preferably short-path distillation.

In addition to the use of a pre-hydrolysis step, the process may be further optimized by accelerating step c by
(i) addition of an additive from the group of weakly basic salts, complexing agents and basic ion exchangers and/or
(ii) addition of an entraining agent in the form of a solvent or a gas and/or
(iii) addition of glycerol-binding adsorbers and/or
(iv) heat treatment of the partial glyceride intermediate product.

The present invention will now be illustrated in more detail by reference to the following specific, non-limiting examples.

### EXAMPLE 1

### Comparison of the Stabilities of Novozym 435 in CLA Methyl Ester and CLA-Free Acid Under Operational Conditions

Glycerol (4 g) and CLA fatty acid (44 g) were weighed into one flask in a molar ratio of 1:3.6 while glycerol (5.5 g) and CLA methyl ester (66 g) were weighed into a second flask in a molar ratio of 1:3.7. After addition of Novozym® 435 (Lipase from Novozymes, Denmark; 4 g in batch 1 and 3 g in batch 2), a vacuum of 20 mbar was applied while stirring with a magnetic stirring fish at a temperature of 60° C. A sample of the oil phase is removed after 48 hours and analyzed by gas chromatography for the percentage of glycerides. Novozym 435 is removed from the CLA triglyceride formed by filtration and returned to the flask and another reaction is started under the same reaction conditions. Reactions are carried out over a period of 8 weeks in this way. The reaction starting with CLA methyl ester is carried out under nitrogen.

Results (see drawing):
(♦)=use of CLA methyl ester
(▪)=use of CLA-free acid

The percentage triglyceride content in the product, based on the sum of di- and triglyceride, is determined. It can clearly be seen that, where the CLA methyl ester is used for the synthesis of CLA triglycerides, the capacity of the enzyme used is exhausted after only 5 weeks whereas the enzyme used for the pure esterification still has 80% of its original capacity after 8 weeks. Where CLA-free acid is used, there was only a relatively small reduction in enzyme activity which is attributable both to deactivation of the enzyme and to relatively serious abrasion of the carrier material.

### EXAMPLE 2

### Selection of Suitable Lipases for the Pre-Hydrolysis of CLA Esters

15 batches each containing 4 g of conjugated linoleic acid ethyl ester and 6 g of water were placed in a closable reaction vessel and simultaneously stirred at room temperature on a multiple stirring plate. 40 mg of commercially obtainable lipases or esterases were added to each of the batches. Samples were taken after 2 hours and 22 hours. The organic phase containing fatty acid ethyl ester and enzymatically hydrolyzed fatty acid were separated and analyzed. The conversion was determined through the acid number. The results are set out in Table 1.

TABLE 1
Selection of suitable lipases and esterases for the process claimed in claim 1

| | | | Acid value | | Conversion | |
|---|---|---|---|---|---|---|
| Enzyme | Microorganism | Manufacturer | 2 h | 22 h | 2 h | 22 h |
| Chirazym L-10 | Alcaligenes sp. | Roche | 21 | 41 | 11.5 | 20.5 |
| Lipase A | Aspergillus niger | Amano | 6 | 16 | 3 | 8 |
| Novozym 868 | Candida antarctica A | Novozymes | 5 | 6 | 2.5 | 3 |
| Novozym 525 | Candida antarctica B | Novozymes | 52 | 62 | 26 | 30 |
| Lipomod 34 | Candida cylindracea | Biocatalysts | 45 | 61 | 22.5 | 30 |
| Lipase LP | Chromobacterium viscosum | Asahi Kasei | 45 | 60 | 22.5 | 30 |
| Novozym 388 | Rhizomucor miehei | Novozymes | 8 | 11 | 4 | 5.5 |
| Lipase G | Penicilium camenberti | Amano | 15 | 38 | 7.5 | 19 |
| Lipase R | Penicilium roqueforti | Amano | 6 | 6 | 3 | 3 |
| Lipase L115P | Porcine pancreas | Biocatalysts | 6 | 6 | 3 | 3 |
| Lipase PS | Pseudomonas cepacia | Amano | 46 | 57 | 23 | 28.5 |
| Lipase AK | Pseudomonas fluorescens | Amano | 26 | 53 | 13 | 26.5 |
| Lipomod 36 P | Rhizopus javanicus | Biocatalysts | 21 | 38 | 11.5 | 19 |
| Lipase F-AP 15 | Rhizopus oryzae | Amano | 12 | 18 | 6 | 9 |
| Lipolase Tl 100 | Thermomyces lanugenosus | Novozymes | 38 | 53 | 19 | 26.5 |

All the lipases and esterases tested were found to be active in the hydrolysis of the fatty acid esters. However, microorganisms of the Alcaligenes, Candida, Chromobacterium, Penicilium, Pseudomonas, Rhizopus and Thermomyces type are preferred. Under the above conditions, the reaction without removal of ethanol continued to an equilibrium of ca. 30% by weight free fatty acid.

### EXAMPLE 3

### Hydrolysis of Short-Chain Conjugated Linoleic Acid Methyl Esters with Continuous Stripping of Water and Methanol

100 g of conjugated linoleic acid methyl ester, 10 g of water and 5 g of immobilized Candida antarctica B lipase were introduced into a heatable flask. With a distillation bridge attached, the reaction was carried out under a reduced pressure of 60 mbar and at a temperature of 60° C. Water was continuously pumped into the flask at a flow rate of 0.25 ml/min. (Example 3A) and 0.5 ml/min. (Example 3B). Water added was quickly distilled off, so that the water content in the reactor was low (<20%) throughout the reaction. The conversion was monitored by determination of the acid value. The reactions were terminated after 24 hours (partial conversion) and the immobilized enzyme was filtered off from the reaction mixture. The organic phase was then separated from the aqueous phase. An acid value of 200 corresponds to a 100% conversion. The results are set out in Table 4.

TABLE 4
Conversion with stripping of water and methanol after different reaction
times

| Reaction | Acid value | Conversion [%] | Acid value | Conversion [%] |
|---|---|---|---|---|
| time [h] | Example 3A | Example 3A | Example 3B | Example 3B |
| 0 | 0 | 0 | 0 | 0 |
| 2 | 51.5 | 25.7 | 62.3 | 31.1 |
| 4 | 71.2 | 35.6 | 84.4 | 42.4 |
| 6 | 87.0 | 43.5 | 100.2 | 50.1 |
| 8 | 100.0 | 50.0 | 112.2 | 56.1 |
| 24 | 153.0 | 76.5 | 167.1 | 83.5 |

Hydrolysis levels of 76.5% and 83.5% were obtained in 24 hours, depending on the quantity of water added. The quantity of distillate after 24 hours was 315 g in Example 3A and 584 g in Example 3B.

### EXAMPLE 4

### Hydrolysis of Short-Chain Conjugated Linoleic Acid Methyl Esters by Multistage Hydrolysis

3 Batches each containing 20 g of conjugated linoleic acid methyl ester based on sunflower oil and 40 g of water were weighed into closed vessels. 1 g of immobilized lipase was then added to each batch and the mixtures were stirred on a magnetic stirring plate for 5 hours at room temperature. The enzyme immobilizates were then filtered off and the organic phase was separated from the aqueous phase. Another 40 g of water were added to the organic phase and the enzyme immobilizates filtered off were re-added to the reaction solution. After reaction overnight at room temperature, the enzyme immobilizates were again filtered off and the organic phase was separated from the aqueous phase. 40 g of water were added to the organic phase and the enzyme immobilizates filtered off were re-added to the reaction solution. After another 5 hours' reaction at room temperature, the reaction was terminated.

The following enzyme immobilizates were used:
5A) Novozym 435
5B) 1 g immobilized Candida antarctica B lipase
5C) 1 g immobilized Thermomyces lanugenosus lipase

The conversion in the individual stages was monitored by determination of the acid value. An acid value of 200 corresponds to a 100% conversion. The results are set out in Table 6.

TABLE 6
Multistage hydrolysis of short-chain conjugated
linoleic acid methyl esters

| | | Conv. | | | | |
|---|---|---|---|---|---|---|
| Reaction | A.V. | [%] | A.V. | Conv. [%] | A.V. | Conv. [%] |
| time [h] | Ex. 5A | Ex. 5A | Ex. 5B | Ex. 5B | Ex. 5C | Ex. 5C |
| 0 | 0 | 0 | 0 | | | 0 |
| Stage 1, | 100.4 | 50.2 | 81.8 | 40.9 | 74.5 | 37.3 |
| after 5 h | | | | | | |
| Stage 2, | 142 | 71 | 127 | 63.5 | 117.2 | 58.6 |
| after 16 h | | | | | | |
| Stage 3, | 165.5 | 82.8 | 154.9 | 77.5 | 152.4 | 76.2 |
| after 5 h | | | | | | |

A.V. = Acid value,
Conv. = Conversion,
Ex. = Example

### EXAMPLE 5

### Partial Hydrolysis of CLA Methyl and Ethyl Esters and Reaction of the Hydrolyzates with Glycerol to Form CLA Triglyceride

300 g of water and 900 g of CLA methyl ester (batch 1) or 940 g of CLA ethyl ester were weighed into 2 flasks. 0.22% of sodium carbonate were added to batch 1. The hydrolysis was started by addition of 100 g of immobilized Candida B lipase (batch 1) or 80 g of immobilized Candida B lipase (batch 2). The partial hydrolysis was carried out at a temperature of 45° C. (internal temperature), under a vacuum of 60 mbar and at a stirrer speed of 300 r.p.m. Water was continuously added at a flow rate of 3 ml/min. during the hydrolysis. Samples of the oil phase were removed and the degree of hydrolysis was determined by determination of the acid value. After 7 hours, hydrolysis was terminated. Quantities of 80 g of glycerol were added to the batches and the glyceride synthesis was carried out in vacuo (60 mbar in batch 1 and 20 mbar in batch 2) at a temperature of 55° C. (internal temperature). Samples of the oil phase were removed and the content of CLA glycerides formed was determined by gas chromatography. The result is expressed as the percentage triglyceride content, based on the sum of di- and triglyceride formed.

Results:

TABLE 7
Course of the partial hydrolysis of CLA methyl and ethyl ester and
reaction of the hydrolyzates with glycerol to form CLA triglyceride

| Reaction time | Batch 1 | Batch 2 |
|---|---|---|
| 3 h | Hydrolysis level 60% | Hydrolysis level 67% |
| 6 h | | Hydrolysis level 84% |
| 7 h | Hydrolysis level 91% | |
| 24 h | 55% Triglyceride | 22% Triglyceride |
| 31 h | 61% Triglyceride | 40% Triglyceride |
| 48 h | 83% Triglyceride | 75% Triglyceride |

High CLA triglyceride yields are obtained by the combined hydrolysis of CLA esters and the glyceride synthesis starting from the partial hydrolyzates. A high hydrolysis level is achieved after only a few hours. In the combined process, too, the addition of sodium carbonate (batch 1) leads to an increased triglyceride formation rate.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A process comprising:
(a) providing a C₁₋₄ alkyl ester of a polyunsaturated fatty acid;
(b) hydrolyzing the C₁₋₄ alkyl ester of a polyunsaturated fatty acid to form the polyunsaturated fatty acid and a C₁₋₄ alkanol;
(c) reacting the polyunsaturated fatty acid and glycerol, under a vacuum and low-water conditions, in the presence of an enzyme, to form a product mixture comprising (i) a triglyceride of the polyunsaturated fatty acid and (ii) one or more other components selected from the group consisting of the enzyme, unreacted polyunsaturated fatty acid, unreacted glycerol, unhydrolyzed C₁₋₄ alkyl ester and mixtures thereof; and
(d) separating the triglyceride and the one or more other components.

2. The process according to claim 1, wherein the hydrolysis is carried out under vacuum with the continuous addition of water.

3. The process according to claim 2, wherein the hydrolysis is carried out under a pressure of 200 mbar or less.

4. The process according to claim 1, wherein the hydrolysis is carried out in two or more stages without vacuum.

5. The process according to claim 1, wherein the process is carried out as a one-pot process in a single reactor.

6. The process according to claim 1, wherein the hydrolysis and the reaction of the polyunsaturated fatty acid and the glycerol are both carried out in the presence of the enzyme.

7. The process according to claim 6, wherein the process is carried out as a one-pot process in a single reactor.

8. The process according to claim 1, wherein the polyunsaturated fatty acid comprises a compound selected from the group consisting of docosahexanoic acid, eicosapentaenoic acid, arachidonic acid, γ-linolenic acid, linoleic acid, conjugated linoleic acid, C₁₋₄ alkyl esters thereof and mixtures thereof.

9. The process according to claim 7, wherein the polyunsaturated fatty acid comprises a compound selected from the group consisting of docosahexanoic acid, eicosapentaenoic acid, arachidonic acid, γ-linolenic acid, linoleic acid, conjugated linoleic acid, C₁₋₄ alkyl esters thereof and mixtures thereof.

10. The process according to claim 1, wherein the enzyme is immobilized on a carrier.

11. The process according to claim 1, wherein the enzyme is selected from the group consisting of lipases, phospholipases, esterases and mixtures thereof.

12. The process according to claim 10, wherein the enzyme is selected from the group consisting of lipases, phospholipases, esterases and mixtures thereof.

13. The process according to claim 12, wherein the polyunsaturated fatty acid comprises a compound selected from the group consisting of docosahexanoic acid, eicosapentaenoic acid, arachidonic acid, γ-linolenic acid, linoleic acid, conjugated linoleic acid, C₁₋₄ alkyl esters thereof and mixtures thereof.

14. The process according to claim 1, wherein water is present during the hydrolysis in an amount of from 1 to 50% by weight.

15. The process according to claim 1, wherein the reaction of the polyunsaturated fatty acid and the glycerol is carried out under a pressure of 200 mbar or less without the addition of water.

16. The process according to claim 13, wherein the reaction of the polyunsaturated fatty acid and the glycerol is carried out under a pressure of 200 mbar or less without the addition of water.

17. The process according to claim 1, wherein the reaction of the polyunsaturated fatty acid and the glycerol is carried out in the further presence of an agent selected from the group consisting of weakly acidic salts, weakly basic salts, complexing agents, salts of complexing agents, basic ion exchangers, weakly basic ion exchangers, salts of acidic ion exchangers, solvent and gas entraining agents, glycerol-binding adsorbers, and mixtures thereof.
